(19) United States
(12) Patent Application Publication
(10) Pub. No.: US 2023/0178212 A1
(43) Pub. Date: Jun. 8, 2023

(54) METHOD AND SYSTEM FOR FOODSERVICE WITH IOT-BASED DIETARY TRACKING

(71) Applicant: SKOOPIN INC., San Jose, CA (US)

(72) Inventors: Fengmin GONG, LOS ALTOS HILLS, CA (US); Weijun ZHANG, SAN JOSE, CA (US); Min FAN, BELLEVUE, WA (US); Jun DU, CUPERTINO, CA (US)

(21) Appl. No.: 17/542,066

(22) Filed: Dec. 3, 2021

(51) Int. Cl. G16H 20/60; G16H 10/20; G06N 20/00; G06V 40/16; G06V 20/68; G16Y 10/60

(52) U.S. Cl. CPC G16H 20/60; G06N 20/00; G06V 20/68; G06V 40/172; G16H 10/20; G16Y 10/60

(57) ABSTRACT

Methods, systems, and apparatuses, including computer programs encoded on computer storage media, for intelligent dietary tracking are described. An example method includes obtaining a complete coverage of a user’s food intake activities using multiple tools. These tools are respectively designed to collect the user’s food intake activities through different venues. For example, the obtaining may include: obtaining first food intake events of the user collected by an Internet of Things (IoT) system installed as a foodservice establishment; obtaining second food intake events of the user collected by an electronic appliance placed at the user’s residence or office; and obtaining third food intake events of the user from a mobile application installed on a mobile device of the user. The complete coverage of the user’s food intake activities may be used for dietary behavioral analysis or other tracking purposes.

### TECHNICAL FIELD

The disclosure generally relates to systems and methods for food servicing, specifically, an Artificial Intelligence (AI) assisted multi-sensor Internet-of-Things (IoT) system for complete tracking of dietary intake of users.

### BACKGROUND

Dietary tracking provides users better understanding of current eating habits, informs users how nutrient-dense the food is, helps balance total calories and macronutrients throughout the day, and more importantly, helps users to reach their goals/nutrient needs. With the tracking information, the users may become mindful of when and what to eat. A complete tracking of dietary intake of users may also benefit public health and health care. For example, should some food poisoning or bacteria contamination occur, the tracking information (records) may provide a powerful tool for source tracing and infection control. By a similar reasoning, the tracking information may be of great importance for doctors to diagnose a patient’s illness or disease related to food habits.

Unfortunately, existing dietary tracking tools, such as mobile or web-based applications, require heavy user involvement and manual work. For instance, a user has to manually enter and upload the food information to a dietary tracking app for tracking his/her diet. In this disclosure, an IoT-based dietary tracking system is described. The IoT-based dietary tracking system provides complete coverage of users’ dietary data collection with maximized automation and offers users dietary analysis reports using machine learning-based data analysis.

### SUMMARY

Various embodiments of the present specification may include systems, methods, and non-transitory computer-readable media for IoT-based dietary tracking.

According to a first aspect, a computer-implemented method for IoT-based dietary tracking is described. The method may include: obtaining a plurality of food intake events of a user occurred at a plurality of venues, wherein the obtaining comprises: obtaining first food intake events of the user collected by an Internet of Things (IoT) system installed as a foodservice establishment; obtaining second food intake events of the user collected by an electronic appliance placed at the user’s residence or office; obtaining third food intake events of the user from a mobile application installed on a mobile device of the user; and generating a dietary analysis report for the user based on the plurality of food intake events of the user.

In some embodiments, the obtaining of the first food intake events may include: determining portion-based dietary information by monitoring the user’s food taking actions using geometrically distributed sensors, wherein the geometrically distributed sensors comprise distributed weight sensors attached to a scale and one or more cameras; generating a notification on a mobile device of the user for confirming an identification of the user; and associating the portion-based dietary information of the first food intake event with the identification of the user to form a first food intake event.

In some embodiments, the electronic appliance may include: a scale coupled with one or more weight sensors, a first camera facing the scale, and a second camera facing users. The obtaining of the second food intake events may include: receiving a first weight signal from the one or more weight sensors when the user places first food on the scale; receiving an image of the first food from the first camera facing the scale; determining food information of the first food based on the image of the first food using a first machine learning model for food image recognition; determining portion-based dietary information of the first food based on the food information of the first food and the first weight signal; receiving an image of the user from the second camera; determining an identification of the user based on the image of the user using a second machine learning model for face recognition; and associating the identification of the user with the portion-based dietary information of the first food to form a second food intake event;

In some embodiments, the obtaining the second food intake events of the user collected by the electronic appliance further comprises: when another user places second food on the scale, receiving an image of the another user from the second camera and a second weight signal from the one or more weight sensors; determining an identification of the another user based on the image of the another user using the second machine learning model; in response to the identification of the another user being the same as the identification of the user, displaying, on a display of the electronic appliance, a prompt for the user to confirm whether the second food is new food or leftover; in response to the second food being leftover, updating the portion-based dietary information based on a difference between the first weight signal and the second weight signal; and in response to the second food being new food, updating the portion-based dietary information based on a sum of the first weight signal and the second weight signal.

In some embodiments, the obtaining the second food intake events of the user collected by the electronic appliance further comprises: when the user places second food on the scale, receiving an image of the second food from the first camera and a second weight signal from the one or more weight sensors; determining whether the second food is same as the first food using the first machine learning model based on the image of the second food; if the second food is the same as the first food, displaying, on a display of the electronic appliance, a prompt for the user to confirm whether the second food is new food or leftover; in response to the second food being leftover, updating the portion-based dietary information based on a difference between the first weight signal and the second weight signal; and in response to the second food being new food, updating the portion-based dietary information based on a sum of the first weight signal and the second weight signal.

In some embodiments, the obtaining the second food intake events of the user collected by the electronic appliance further comprises: in response to determining that the second food is different from the first food, updating the portion-based dietary information based on the sum of the first weight signal and the second weight signal.

In some embodiments, the obtaining of the second food intake events of the user collected by the electronic appliance further comprises: associating the identification of the user, the portion-based dietary information of the first food, and a current timestamp to form the second food intake event.

In some embodiments, the method may further include: correlating the plurality of food intake events based on the identification of the user associated with the plurality of food intake events.

In some embodiments, the generating of the dietary analysis report for the user comprises: receiving a request comprising a time window; and generating a list of food intake events of the user between the time window, wherein each food intake event comprises a time, a location, and portion-based food information of food taken by the user.

In some embodiments, the generating of the dietary analysis report for the user comprises: obtaining a plurality of historical food intake events of a plurality of users; determining, using feature selection techniques in machine learning, a set of dietary features of each of the plurality of users based on the plurality of historical food intake events; clustering, using unsupervised learning, the plurality of users into a plurality of dietary behavioral groups based on the set of dietary features of each of the plurality of users; obtaining a plurality of group labels for the plurality of dietary behavioral groups; training, using supervised training, a classification model based on the plurality of group labels and the set of dietary features; classifying, using the classification model, the user into one of the plurality of dietary behavioral groups based on the set of dietary features of the user extracted from the plurality of food intake events of the user; and generating the dietary analysis report for the user based on the set of dietary features of the user and other users in the classified dietary behavioral group.

In some embodiments, the generating of the dietary analysis report for the user comprises: receiving a plurality of historical dietary goals from a plurality of users; clustering the plurality of users based on the plurality of historical dietary goals into a plurality of dietary goal groups; and for each of the plurality of dietary goal groups, determining representative dietary features values for the dietary goal group based on a set of dietary features of the users in the dietary goal group.

In some embodiments, the generating of the dietary analysis report for the user comprises: receiving a dietary goal from the user; identifying one of the plurality of dietary goal groups to which the dietary goal of the user belongs; determining a distance between the set of dietary features of the user and the representative feature values of the identified dietary goal group; and generating the dietary analysis report for the new user based on the distance.

In some embodiments, the determining the set of dietary features of each of the plurality of users based on the plurality of historical food intake events comprises: determining a plurality of features based on the plurality of historical food intake events of the plurality of users; determining a correlation coefficient between each pair of the plurality of features; grouping the plurality of features based on the correlation coefficients into one or more groups; and selecting one feature from each of the one or more groups to form the set of dietary features.

In some embodiments, the obtaining the first food intake event of the user collected by the IoT system installed at the foodservice establishment comprises: receiving the first food intake event of the user from the IoT system, wherein the first food intake event comprises a time, a location, portion-based food information of food taken by the user at the foodservice establishment, and an identification of the user.

In some embodiments, the obtaining the third food intake events comprises: installing an application on the mobile device of the user, wherein the application comprises a trained machine learning model that is trained to receive a food image and output one or more predicted food images that are similar to the food image; and receiving, from the application, a third food intake event comprising a time, a user selection of the one or more predicted food images generated by the trained machine learning model, and an identification of the user.

According to a second aspect, an IoT-based dietary tracking system is described. The system may be configured with one or more processors and one or more non-transitory computer-readable memories coupled to the one or more processors and configured with instructions executable by the one or more processors to cause the system to perform various operations. In some embodiments, the operations include: collecting first food intake events of the user using an Internet of Things (IoT) system installed as a foodservice establishment, wherein the collecting comprises: determining portion-based dietary information by monitoring the user’s food taking actions using geometrically distributed sensors, wherein the geometrically distributed sensors comprise distributed weight sensors attached to a scale and one or more cameras; generating a notification on a mobile device of the user for confirming an identification of the user; and associating the portion-based dietary information of the first food intake event with the identification of the user to form a first food intake event; collecting second food intake events of the user using an electronic appliance placed at the user’s residence or office, wherein the electronic appliance comprises: a scale coupled with one or more weight sensors and a first camera facing the scale, and wherein the collecting comprises: receiving a first weight signal from the one or more weight sensors when the user places first food on the scale; receiving an image of the first food from the first camera facing the scale; determining food information of the first food based on the image of the first food using a first machine learning model for food image recognition; determining portion-based dietary information of the first food based on the food information of the first food and the first weight signal; determining the identification of the user based on the user’s biometric features or the user’s selection from a list of user profiles; and associating the identification of the user with the portion-based dietary information of the first food to form a second food intake event; collecting third food intake events of the user using a mobile application installed on the mobile device of the user; correlating the plurality of food intake events based on the identification of the user associated with the plurality of food intake events; and generating a dietary analysis report for the user based on the plurality of food intake events of the user.

According to a third aspect, a non-transitory computer-readable storage medium is described. The storage medium may be configured with instructions executable by one or more processors to cause the one or more processors to perform operations including: obtaining a plurality of historical food intake events of a plurality of users, the plurality of historical food intake events comprising: obtaining a plurality of food intake events of a user, wherein the obtaining comprises: collecting first food intake events of the user using an Internet of Things (IoT) system installed as a foodservice establishment, wherein the collecting comprises: determining portion-based dietary information by monitoring the user’s food taking actions using geometrically distributed sensors, wherein the geometrically distributed sensors comprise distributed weight sensors attached to a scale and one or more cameras; generating a notification on a mobile device of the user for confirming an identification of the user; and associating the portion-based dietary information of the first food intake event with the identification of the user to form a first food intake event; collecting second food intake events of the user using an electronic appliance placed at the user’s residence or office, wherein the electronic appliance comprises: a scale coupled with one or more weight sensors and a first camera facing the scale, and wherein the collecting comprises: receiving a first weight signal from the one or more weight sensors when the user places first food on the scale; receiving an image of the first food from the first camera facing the scale; determining food information of the first food based on the image of the first food using a first machine learning model for food image recognition; determining portion-based dietary information of the first food based on the food information of the first food and the first weight signal; determining the identification of the user based on the user’s biometric features or the user’s selection from a list of user profiles; and associating the identification of the user with the portion-based dietary information of the first food to form a second food intake event; collecting third food intake events of the user using a mobile application installed on the mobile device of the user; correlating the plurality of food intake events based on the identification of the user associated with the plurality of food intake events; and generating a dietary analysis report for the user based on the plurality of food intake events of the user.

These and other features of the systems, methods, and non-transitory computer-readable media disclosed herein, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for purposes of illustration and description only and are not intended as a definition of the limits of the invention.

### BRIEF DESCRIPTION OF THE DRAWINGS

FIG. 1 illustrates an exemplary environment for IoT-based dietary tracking in accordance with some embodiments.

FIG. 2A illustrates an exemplary IoT system installed at a foodservice establishment for IoT-based dietary tracking in accordance with some embodiments.

FIG. 2B illustrates an exemplary system diagram of an IoT system installed at a foodservice establishment for IoT-based dietary tracking in accordance with some embodiments.

FIG. 3 illustrates an exemplary portable IoT device for IoT-based dietary tracking in accordance with some embodiments.

FIG. 4 illustrates an exemplary method of a mobile application for IoT-based dietary tracking in accordance with some embodiments.

FIG. 5A illustrates an exemplary system diagram for IoT-based dietary tracking in accordance with some embodiments.

FIG. 5B illustrates another exemplary system diagram of a cloud server for IoT-based dietary tracking in accordance with some embodiments.

FIG. 6 illustrates an exemplary method for IoT-based dietary tracking in accordance with some embodiments.

FIG. 7 illustrates an example computing device in which any of the embodiments described herein may be implemented.

### DETAILED DESCRIPTION

The description is presented to enable any person skilled in the art to make and use the embodiments, and is provided in the context of a particular application and its requirements. Various modifications to the disclosed embodiments will be readily apparent to those skilled in the art, and the general principles defined herein may be applied to other embodiments and applications without departing from the spirit and scope of the present specification. Thus, the specification is not limited to the embodiments shown but is to be accorded the widest scope consistent with the principles and features disclosed herein.

In this disclosure, an AI-assisted Internet of Things (IoT) system is described to track users’ dietary data in an end-to-end manner, provide complete food intake monitoring, apply correlation analysis using machine learning algorithms for dietary insights, and make recommendations to users based on the analysis. This IoT system interrelates computing devices, mechanical and digital sensors and machines, objects, and the ability to transfer data over a network without requiring human-to-human or human-to-computer interaction.

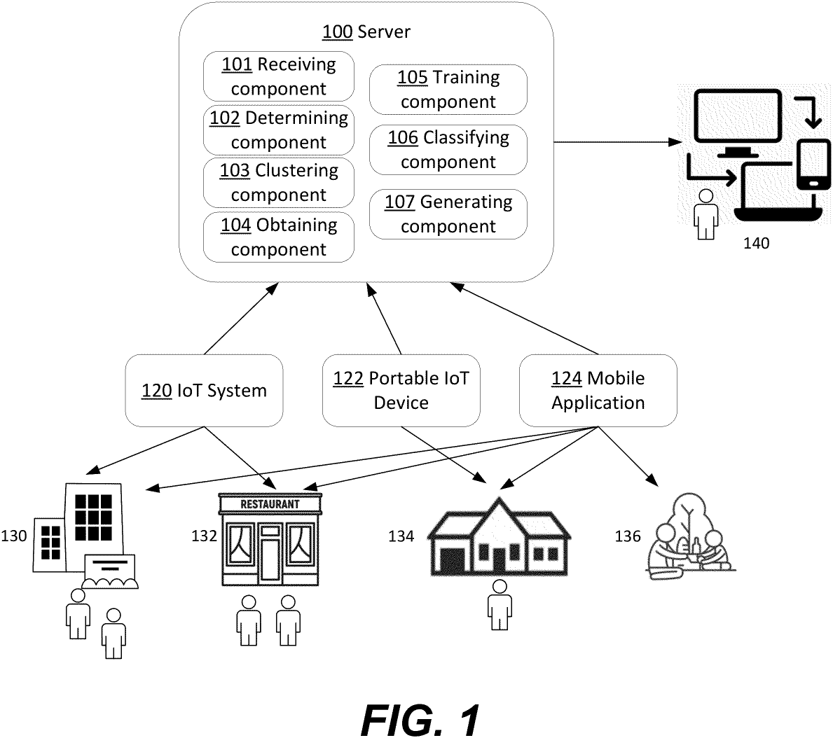

FIG. 1 illustrates an exemplary environment for IoT-based dietary tracking in accordance with some embodiments. The components and parties shown in FIG. 1 are for illustrative purposes only. The exemplary environment in FIG. 1 lists multiple venues for people to perform food-intaking activities. For instance, companies, schools, convention centers, or hotels may have internal foodservice establishments such as cafeterias to serve employees, students/staff, or conference attendees. As another example, people may also eat at restaurants 132, homes 134, offices 130, or outdoor places 136. Different venues may have different features, and thus different devices may be developed and used to collect users’ food intake data.

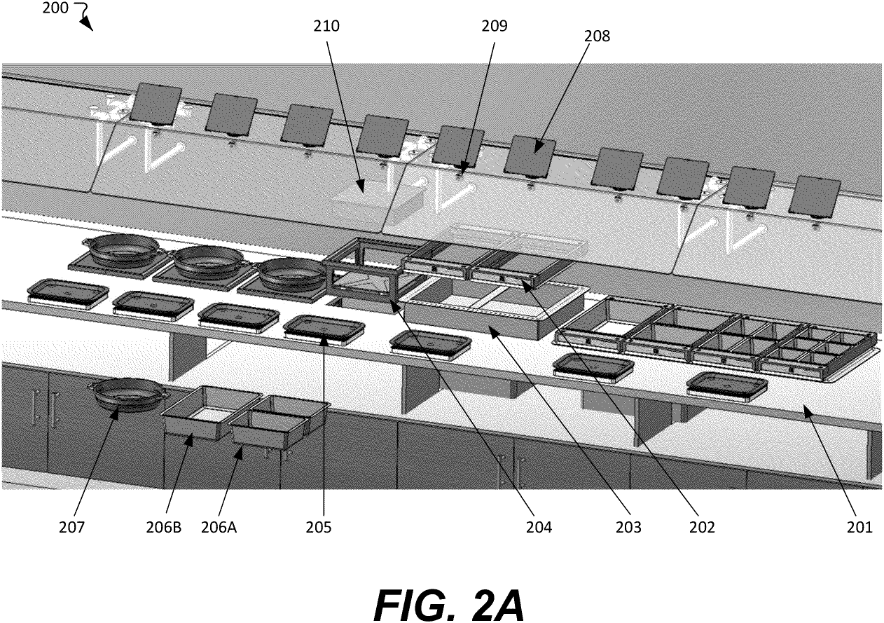

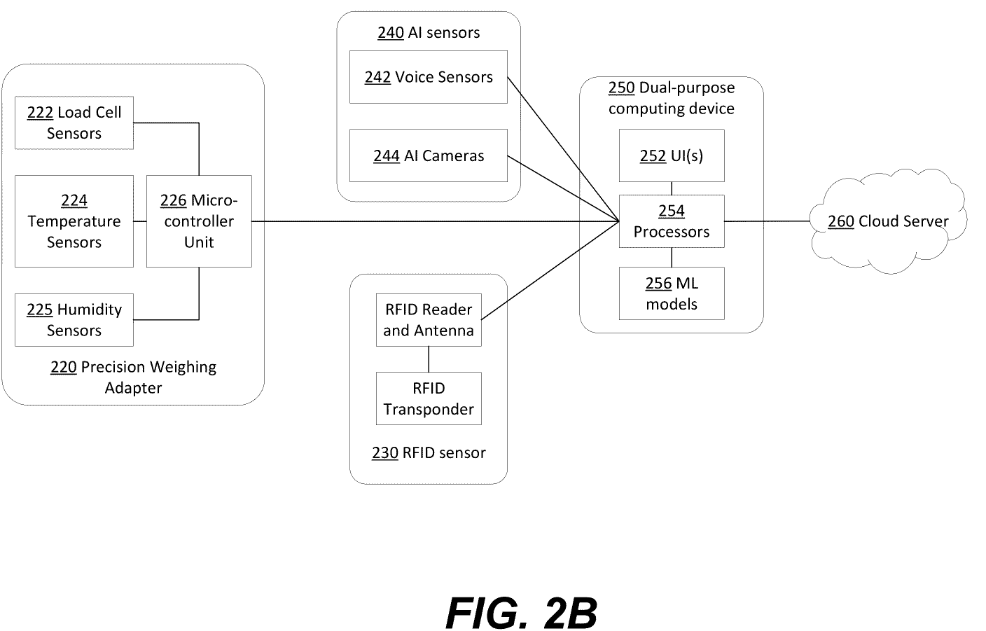

In some embodiments, an IoT system 120 may be deployed at foodservice establishments, such as a cafeteria of a building 130, a buffet of a restaurant 132, or other suitable settings, to automatically and accurately monitor the food taken by a user, collect food information (e.g., amount/weight, nutrition information, allergen information of the food taken by the user), associate the food information with the user, and send a record of food intake event to a server 100 for data aggregation and further analysis. In some embodiments, the record of food intake event includes a time, the portion-based food information of the food taken by the user (e.g., specifically measured based on the portion that the user has taken), and an identification of the user (e.g., a user ID obtained based on face-recognition or scanning a badge or tag). The IoT system 120 may include multiple hardware IoT devices such as a smart frame equipped with weight sensors, AI cameras, radio-frequency identification (RFID) readers and antennas, RFID-equipped food trays, displays, on-prem servers, etc. These IoT devices may be installed at different places within the food establishment, and thus suitable for places with stationary configurations. A detailed description of the IoT system 120 may be found in FIGS. 2A and 2B.

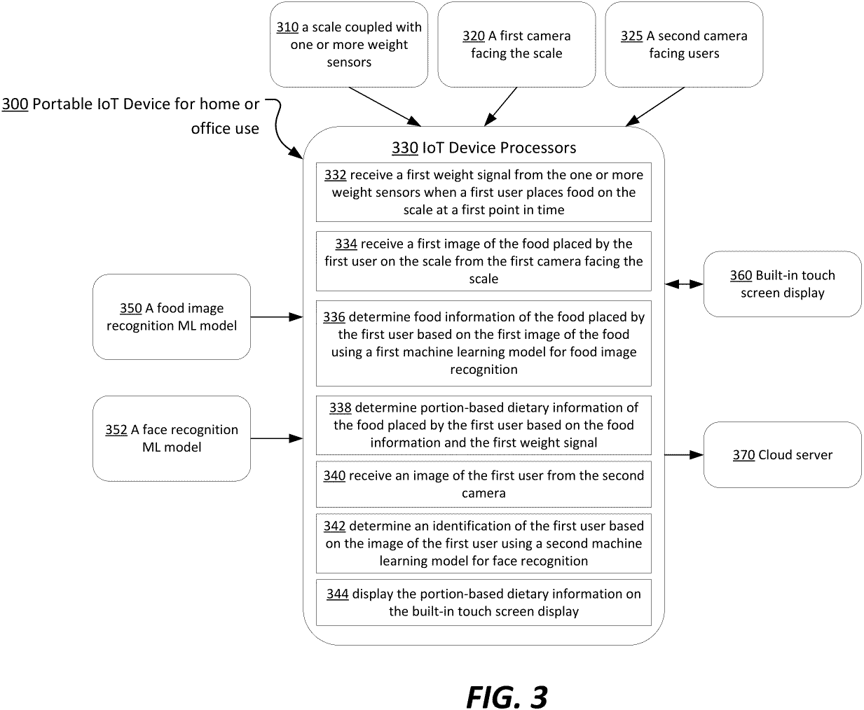

In some embodiments, a portable IoT device 122 may be designed to monitor a user’s food intake activities at home or office where installing the IoT system 120 is difficult. The portable IoT device 122 may refer to an integrated electronic device (like a coffee machine) for home/office use. In some embodiments, the portable IoT device 122 may include multiple integrated hardware pieces, such as a scale coupled with one or more weight sensors, a first camera facing the scale for taking images of food, a second camera facing users, a built-in touch screen display for I/O purposes, and a build-in computing device with one or more processors to process collected data. The portable IoT device 122 may allow a user to scan (e.g., by using the scale and the first camera) the food before and after consumption and determine the accurate portion-based nutrition information. The IoT device 122 may also associate the information with the user and send it to the server 100 for data aggregation and further analysis. A detailed description of the portable IoT device 122 may be found in FIG. 3.

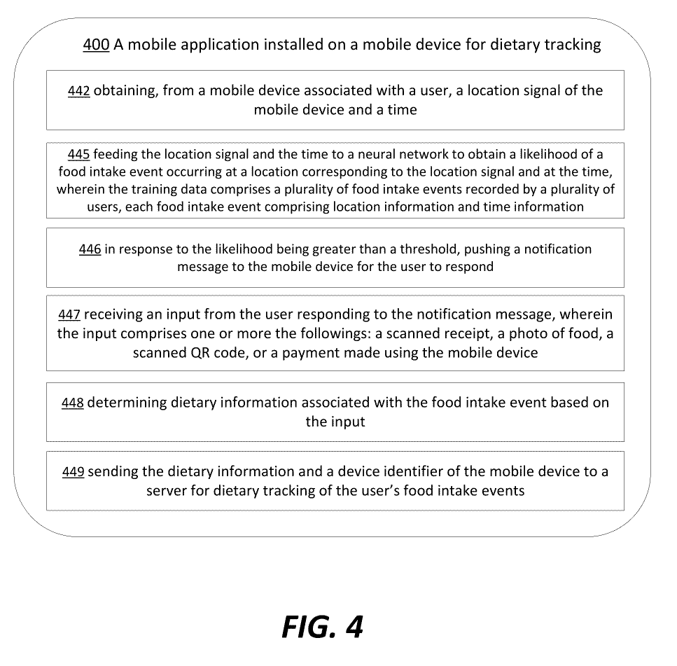

In some embodiments, when the venue is not suitable for installing the IoT system 120 or using the portable IoT device 122, a mobile application 124 installed on a mobile device may be used for tracking a user’s food intake activity. These venues may include outdoor dining/picnic, food trucks, restaurants without the IoT system 120, or other places. The mobile device may include a smartphone, a tablet, a laptop, a wearable smart device, or another suitable device that is carried by the user. In some embodiments, the mobile application 124 may include trained machine learning models for recognizing food based on images and automatically determining the nutrition information. The mobile application 124 may also allow users to fine-tune the nutrition information, and send such information to the server 100 for data aggregation and further analysis. For example, the mobile application 124 may receive an image of the user’s food captured by the user using a camera of the mobile phone, feed the food image into the trained machine learning model to generate one or more predicted food images (sharing similarities with the user’s food), and display these predicted food images for the user to select the matching food and further fine-tune the matching food. The fine-tuning step may include adding/removing/changing ingredients, changing the portion size, etc. A detailed description of the mobile application 124 may be found in FIG. 4.

In some embodiments, the server 100 may act as a centralized hub for aggregating and processing the food-intake data collected by the IoT systems 120, the portable IoT devices 122, and the mobile applications 124. It is to be understood that the server 100 is shown in FIG. 1 as a single entity, this is merely for ease of reference and is not meant to be limiting. The server 100 may be implemented with any number of interconnected computing devices, which may be implemented in one or more networks (e.g., enterprise networks), one or more endpoints, one or more servers, or one or more clouds. The server 100 may include hardware or software which manages access to a centralized resource or service in a network. The server 100 may refer to one or more computing devices in a cloud. The server 100 may communicate with IoT systems 120, portable IoT devices 122, and mobile applications 124 over the internet.

In some embodiments, the server 100 may include a receiving component 101, a determining component 102, a clustering component 103, an obtaining component 104, a training component 105, a classifying component 106, and a generating component 107. The components listed in FIG. 1 are for illustrative purposes only. The server 100 may include fewer, more, or alternative components depending on the implementation.

In some embodiments, the receiving component 101 may be configured to receive a plurality of historical food intake events of a plurality of users. The plurality of historical food intake events may include: a first food intake event of a user collected by the IoT system 120 installed at a foodservice establishment; a second food intake event collected by the IoT device 207 at the user’s residence or office; and a third food intake event collected by the mobile application 208 on a mobile device of the user. These data collecting systems/devices/applications are designed to provide complete coverage of a user’s dietary data collection with maximum automation.

In some embodiments, the determining component 102 may be configured to determine a set of dietary features of each of the plurality of users based on the plurality of historical food intake events using feature selection techniques in machine learning. The purpose of this feature determination step is to cluster the plurality of users into groups, and the users within the same group may share similar dietary behavior (represented by the selected features). Some example features may include a food-intake frequency of the user, portion-based nutrition information of each food-intake event of the user, and timestamps of the plurality of food-intake events of the user. In some embodiments, various features may be extracted from the plurality of historical food intake events of a user, but not all features are equally informative for clustering the users. Machine learning-based feature selection techniques may be used to identify the most distinguishable features. For example, a correlation coefficient between each pair of the various features may be computed, and the various features may be grouped based on the correlation coefficients into one or more groups. The features with similar correlation coefficients (e.g., the difference is within a threshold value) may be grouped together. Subsequently, a representative feature may be selected from each of the one or more groups to form the set of features, which may be used for clustering the users.

In some embodiments, the clustering component 103 may be configured to cluster the plurality of users into a plurality of dietary behavioral groups based on the set of dietary features of each of the plurality of users by using unsupervised machine learning. Unsupervised machine learning works with training data that are not labeled. The clustering with unsupervised machine learning is to find structures or patterns in a collection of uncategorized data, i.e., the set of dietary features of each user. That is, users with similar dietary behavior (reflected by the set of dietary features) are clustered as one group. In some embodiments, the number of features in the set of features for clustering defines the number of dimensions of the space in which the users’ dietary features are distributed. Performing clustering at high dimensions (e.g., four dimensions if four features are used for clustering) cannot be practically performed by the human mind. Machine learning algorithms, such as hierarchical clustering, K-means clustering, or K nearest neighbors, may be used to execute the user clustering in the multi-dimensional space.

In some embodiments, the obtaining component 104 may be configured to a plurality of group labels for the plurality of dietary behavioral groups. For example, for each of the plurality of dietary behavioral groups, one or more profiles of one or more users in the dietary behavioral group may be obtained (e.g., at the time of user registration). Based on the one or more profiles, a label may be generated to represent the dietary behavioral group. In some embodiments, the labels may be generated based on knowledge-based rules. For example, if the food intake of a group of users is featured with high calorie and protein, low sugar and fruit, more than three meals a day, this group of people may be labeled as a group doing regular weight training and bodybuilding. The labels may be sent to some of the users for confirmation or revision.

In some embodiments, the training component 105 may be configured to train a classification model based on the plurality of group labels and the set of dietary features using supervised training. Different from the above-described unsupervised learning-based clustering, the supervised training here refers to training a model for classification based on labeled training data, i.e., the group labels and the set of features of each user in each dietary behavioral group. In some embodiments, the classification model may be a neural network, a decision tree, a logistic regression model, a random forest, or another suitable model. The training may include: feeding the set of features of a user into the model to obtain an output label; determining a distance between the output label and the group label of the group to which the user belongs; and adjusting the weights or parameters of the model to minimize the distance.

In some embodiments, the classifying component 106 may be configured to classify a new user into one of the plurality of dietary behavioral groups using the trained classification model. The classification may be based on the set of dietary features of the new user extracted from a plurality of food intake events of the new user.

In some embodiments, the generating component 107 may be configured to generate a list of food intake events of a user between a time window as requested. Each food intake event may include a time, a location, and portion-based food information of food taken by the user. This list may be treated as a complete coverage of the user’s food-intake activities. The list may be used for public health and health care purposes. For example, should some food poisoning or bacteria contamination occur, the list may provide a powerful tool for source tracing and infection control. By a similar reasoning, the list may be of great importance for doctors to diagnose a patient’s illness or disease related to food habits. In some embodiments, the list of food intake events may be embedded as points in a multi-dimensional space, with one dimension corresponding to time, one dimension corresponding to location, one or more dimensions corresponding to various nutrition intakes, etc. By analyzing the trend of the points in the multi-dimensional space, one user’s dietary behavioral changes, anomalies, patterns may be generated.

In some embodiments, the generating component 107 may be further configured to generate a dietary analysis report to the new user based on the set of dietary features of the new user and other users in the classified dietary behavioral group. For example, average dietary feature values of the classified dietary behavioral group may be computed based on the set of dietary features of the other users in the group. The dietary features of the new user may be compared against the average dietary feature values to obtain one or more distances. Each distance may represent a quantified difference between the new user’s one dietary aspect and a popular average. The dietary analysis report may list the dietary aspects if the corresponding quantified differences are greater than a preset threshold.

In some embodiments, besides the dietary features of the users, the server 100 may also obtain a plurality of dietary goals set by the users. Based on the dietary goals, the users may be clustered into different dietary goal groups, each group consisting of the users sharing the same dietary goal. For each of the plurality of dietary goal groups, representative dietary features values for the dietary goal group may be determined based on the set of dietary features of the users in the dietary goal group. The representative dietary features values may reflect the average dietary behavior of people targeting the same dietary goal. When a new user submits his/her dietary goal, he/she may be classified into one of the dietary goal groups. The dietary features of the new user may be compared against the representative dietary features values of people with the same dietary goal. The comparison result may include multiple distances, which may be used to generate the dietary analysis report.

In some embodiments, the dietary analysis report may be transmitted to the corresponding user via emails or messages to his smart devices 140 like a smart watch, smart phone, tablet, or computer.

In summary, the complete coverage of each user’s dietary activities may be used for analyzing user’s dietary behavior from two different perspectives: (1) vertical analysis: a user’s historical current dietary data is compared against the user’s historical dietary data to find behavior changes, anomalies, patterns, etc.; and (2) horizontal analysis: different users’ dietary behavioral data are compared to find common behaviors of a group, identify outliers to detect behavior anomalies.

FIG. 2A illustrates an exemplary IoT system installed at a foodservice establishment 200 for IoT-based dietary tracking in accordance with some embodiments. The establishment 200 is intended to illustrate a foodservice providing station, which may be found in a cafeteria, a buffet, or other suitable settings in which a user (a customer) serves him/herself or is served by an operator (an employee of the foodservice provider). The various configurations in the establishment 200 are merely for illustrative purposes and do not limit the application of the to-be-described IoT system to other suitable configurations or other environments.

As shown in FIG. 2A, station 201 may include a plurality of openings for hosting food wells 203 and/or induction cookers or warmers 210. The food well 203 may host food containers such as Bain-marie pans 206A or 206B. The food well 203 may be hot or cold in order to keep the food in the pans 206A or 206B warm or cold. Each food well may host one or more pans at a time, and each pan 206A or 206B may have different configurations. For example, pan 206A has two separate compartments or sections, while pan 206B has only one compartment or section. The induction cooker or warmer 210 may host a food container such as a pan 207 to keep the food warm.

In the foodservice establishment 200, an IoT system may be deployed to automate the foodservice experience and provide precise portion control and instant feedback to the users. In some embodiments, the IoT system may include a frame 202 or 204 coupled with one or more weight sensors such as load cell sensors, a radio-frequency identification (RFID) reader 205 configured to read tag data from an RFID tag associated with a user account, one or more sensors 209 for detecting and identifying user actions, and one or more computing devices (not shown in FIG. 2A).

In some embodiments, the one or more load cell sensors may be attached to (e.g., on the upper surface or the lower surface) or built-in the frame 202 or 204 to monitor the weight of the food container directly or indirectly placed on top of the frame 202 or 204. The number and placement of load cells may be customized according to the actual use. For example, frame 204 may receive the induction cooker or warmer 210, which hosts a single-compartment food container 207. In this case, frame 204 may be coupled to one load cell at the center of the bottom to monitor the weight of the food in the food container 207.

As another example, frame 202 may receive a multi-compartment pan 206A, and the multiple compartments in the pan 206A may store different food. Since the IoT system is to provide as much information about the food being taken by a user from the pan 206A, the frame 202 may be configured with multiple load cells to, not only monitor the weight changes of the pan 206A, but also approximately identify the compartment from which the food was being taken based on the weight sensor data collected by the load cells. Based on the identified compartment and the food information of the different food stored in the different compartments, the IoT system may obtain more accurate information of the food taken by the user. In some embodiments, the food information may be received from user input (e.g., input by an employee or an operator), or learned by machine learning models based on 3D images/videos captured by cameras.

In some embodiments, the RFID reader 205 may be installed at a proper place on station 201 for users to scan RFID tags. For example, the RFID tag may be attached to or built-in a food tray. When a user places the food tray on the RFID reader 205, the tag data read from the RFID tag may be deemed as an identifier associated with a user account corresponding to the user. As another example, the RFID tag may be built in an employee badge, a card, a wristband, a mobile phone, a smartwatch, or another suitable device or object that is associated with the user. In some embodiments, all information related to the food taken by a user at a first section of the station 108 may be stored and associated with the user (e.g., with its user account), and displayed on a display panel 208. When the user moves to a second section of station 201 to take food, the previously stored information may be retrieved and displayed along with the food that the user just took from the second station. This way, the user can view all information of the list of food that he or she has taken so far.

In some embodiments, the IoT system may include a plurality of display panels respectively corresponding to the RFID readers. When an RFID reader 205 reads the tag data from an RFID tag associated with a user (e.g., via the food tray held by the user or a badge/card possessed by the user), the information of the food that the user has taken so far may be displayed at the display panel corresponding to the RFID reader 205. In other embodiments, the display panel may refer to a display of the user’s smart device (e.g., phone, watch, glasses). That is, the information of the food that the user has taken so far may be displayed on the user’s smart device.

In some embodiments, the RFID reader 205 may include a reader and an antenna that are electrically connected. The reader may be installed anywhere on station 201, while the antenna may be installed at the place labeled as 205 in FIG. 2A.

In some embodiments, the one or more sensors 209 may include motion sensors, light sensors, depth-sensing sensors, time of flight (ToF) cameras, or Light Detection and Ranging (LiDAR) cameras, and other suitable sensors. For example, a motion sensor or a light sensor may be installed corresponding to one foodservice section (e.g., a section hosting a food well 203 or an induction cooker or warmer 210) of station 201. The motion sensor or the light sensor may detect user actions including a user approaching or leaving the foodservice section. These user actions may compensate for the weight sensor data collected by the load cell sensors on the frame (202 or 204) to determine the amount of food taken by a user.

For example, the load cell sensors on the frame (202 or 204) may continuously collect weight readings at millisecond intervals before, during, and after a user takes food from the food container (206A, 206B, or 207). The sequence of continuous weight readings may be constructed as a weight fluctuation pattern, such as a weight-changing curve in the time dimension. The user actions detected by the motion sensor or the light sensor may help determine the start and completion of a user’s food-taking action on the weight-changing curve. This is helpful to improve the accuracy of the weight measurement especially when the food container is subject to fluid sloshing (e.g., when the food is in a liquid form), material agitation or mixing (e.g., stirring), internal chemical reactions (e.g., caused by the heat from the grill, or frying pan, and the toaster, breaks down food’s proteins into amino acids), vibration caused by surrounding environment (e.g., loud music or vibration bass), or other situations where the weight readings of the food container may not be stabilized.

While the motion or light sensors may detect simple user actions such as a user approaching or leaving station 201, in some embodiments, more sophisticated AI cameras may be deployed as the sensors 209 to capture more informative visual information. For example, ToF cameras or LIDAR cameras may be used to capture 3D point cloud images of station 201 and user actions. A ToF camera is a range imaging camera system employing time-of-flight techniques to resolve distance between the camera and the subject for each point of the image, by measuring the round trip time of an artificial light signal provided by a laser or an LED. ToF cameras are part of a broader class of scanner-less LIDAR, in which the entire scene is captured with each laser pulse, as opposed to point-by-point with a laser beam such as in scanning LIDAR systems.

In some embodiments, the AI cameras deployed as sensors 209 may be configured to monitor the foodservice sections on station 201. For example, each AI camera may monitor one or more food containers (206A, 206B, and 207). In some embodiments, the AI camera may capture 3D images of the food container and the user operating at the food container.

For example, the 3D images captured by the AI cameras covering the food container may be fed into various pre-trained machine learning models to identify (1) the configuration of the food container (e.g., a number of compartments in the food container, a layout of the compartments in the food container), (2) status of the food in the food container (e.g., the remaining amount of food in each compartment in the food container), (3) the name or identification of the food in each compartment in the food container, (4) the food ingredients in each compartment in the food container. As an example, the layout of the compartments in the food container may be identified by a machine learning model trained based on labeled images covering all known food container configurations (e.g., usually between 1 to 8 compartments). Based on the layout of the compartments and the weight readings from the load cells, the IoT system may more accurately determine the specific compartment from which the food was taken and the amount of food that was taken. These machine learning models may include knowledge-based (e.g., rule-based) models, or neural networks or decision trees trained based on labeled training data.

As another example, the 3D images captured by the AI cameras covering users operating at the food container may be fed into another machine learning model to identify user actions. This machine learning model may be implemented as neural networks trained with supervised learning methods and based on labeled training data. The user actions may include: lifting a serving utensil from a first compartment within a multi-compartment food container, taking food using the serving utensil from the first compartment, dropping food back to the first compartment, stirring food, placing the serving utensil back to the first compartment of the multi-compartment pan, or placing the serving utensil back to a second compartment of the multi-compartment pan, etc. In some embodiments, these identified user actions may compensate for the weight readings collected by the load cells on the frame (202 or 204) in determining the weight fluctuation pattern. For example, if it is detected that the user moves the serving utensil from one compartment to another compartment, the weight changes caused by the serving utensil movement may be taken into consideration to adjust the estimated weight of the food taken by the user (e.g., the weight reduction at the first compartment minus the weight addition at the second compartment may be determined as the weight of food taken by the user).

As yet another example, the 3D images captured by the AI cameras covering the food being served in the food container may be used for food identification. By identifying the food, detailed food information may be retrieved. Such detailed food information may include name, ingredient information, nutrition information, pricing information, and/or allergen information. Based on the detailed food information and the estimated weight of the food taken by a user, the IoT system may provide customized information for the user, such as the calories/sugar/ carbohydrate amount contained in the portion taken by the user. In some embodiments, the customized information may be analyzed by a corresponding mobile application or a server, which may make recommendations on adjusting the food (e.g., reducing certain ingredients, adding other ingredients, reducing/adding amount).

In some embodiments, the one or more computing devices of the IoT system provide the computation power and storage capacity for implementing the above-described functionalities. The computing devices may include on-prem servers, remote servers, cloud servers, and may be implemented in one or more networks (e.g., enterprise networks), one or more endpoints, one or more data centers, or one or more clouds. The computing devices may include hardware and/or software that manages access to a centralized resource or service in a network. In some embodiments, the computing devices may be electronically coupled to the load cell sensors on the frames (202 and 204) through micro-controller units (e.g., a small computer on a single metal-oxide-semiconductor integrated circuit chip), the RFID readers 205 via BLUETOOTH™ or USB, the sensors 209 via wired or wireless connections, and the display panels 208.

FIG. 2B illustrates an exemplary system diagram of an IoT system installed at the foodservice establishment 200 for IoT-based dietary tracking in accordance with some embodiments. The system diagram may be appreciated as an architecture of the IoT system illustrated in FIG. 2A. The components of the IoT system in FIG. 2B are for illustrative purposes, which may include fewer, more, or alternative components depending on the implementation.

In some embodiments, the IoT system may include five groups of hardware devices as shown in FIG. 2B: a precision weighing adapter 220 (corresponding to the frame 202 and 204 in FIG. 2A), AI sensors 240 (corresponding to the sensors 209 in FIG. 2A), RFID sensor 230 (corresponding to the RFID reader/antenna 205 in FIG. 2A), a dual-purpose computing device 250, and a cloud server 260.

In some embodiments, the precision weighing adapter 220 may include one or more load cell sensors 222, temperature sensors 224, and humidity sensors 225. The number and layout of the load cell sensors 222 may be configured according to the type of food container that the precision weighing adapter 220 supports. For example, the precision weighing adapter 220 may be a rectangular shape frame with an opening and is configured to receive a rectangular food container when the food container is lowered into the opening of the frame. In this case, four load cell sensors 222 may be distributed at four corners of the frame to monitor the weight changes occurring at the four corners of the food container. As another example, if the precision weighing adapter 220 is configured to support a single-compartment food container serving one type of food, one load cell sensor 222 may be sufficient to monitor the weight change of the food in the food container. In some embodiments, the temperature sensors 224 and the humidity sensors 225 may be installed to compensate/adjust the weight readings of the load cell sensors 222. In some embodiments, a micro-controller unit 226 may be installed on the precision weighing adapter 220 for aggregating and preprocessing the collected sensor data, as well as providing communication means to transmit the sensor data to other components of the IoT system.

Besides the sensors on the precision weighing adapter 220, one or more AI sensors 240 may be installed to provide other sensing information from other dimensions. For example, while the sensor data collected by the precision weighing adapter 220 are mainly weight information, an AI camera 244 may be installed to capture visual information related to the food container supported by the precision weighing adapter 220 (e.g., the number and layout of compartments in the food container, food information of the food stored in each of the compartments), and user actions including users approaching or leaving the food container, hand motions such as lifting and putting back a serving utensil (ladle, scoop, pasta server, tong, spatula, etc.), taking food from one of the compartments in the food container, etc. These user actions may be fed into corresponding machine learning models to extract action information, which may be used to compensate or consolidate with the weight information collected by the precision weighing adapter 220. In some embodiments, voice sensors 242 may be installed as an input device for users or operators (e.g., employees of the foodservice provider) to give commands to the IoT system, or as an alternative or additional output interface to the display panel 208 in FIG. 2A.

In some embodiments, RFID sensors 230 may be configured to obtain user information so that the information extracted from the precision weighing adapter 220 and the AI sensors 240 may be associated with the user, thereby providing instant and personalized feedback to the user. In some embodiments, the RFID sensor 230 may include three parts: RFID reader, antenna, and transponder. The transponder refers to an RFID tag that is associated with the user, such as an RFID tag installed on a food tray held by the user, or a badge carried or worn by the user. The RFID reader and antenna are configured to read tag data from the RFID tag. The tag data may be deemed as an identifier corresponding to the user. In some embodiments, the identifier may refer to a user account (e.g., a temporary account just for the current service) created for the user.

In some embodiments, the IoT system may install other types of user identification means, such as Quick Response (QR) code scanner, Near Field Communication (NFC) reader, and other suitable means. For instance, a food tray may have a QR code as an alternative for the RFID tag. If the RFID tag is damaged or the RFID reader/antenna is malfunctioning, the IoT system may trigger the QR code scanner to identify the user or the user account.

In some embodiments, the dual-purpose computing device 250 may refer to one or more computing devices configured with processors 254 for processing the sensor data collected by the precision weighing adapter 220, the AI sensors 240, and the RFID sensor 230, and generating interactive user interface (UI) 252 to present information the users and receiving input from the users. For instance, after identifying a user’s user account based on the data collected by the RFID sensor 230, the processors 254 may retrieve the food information of the food that has been taken by the user so far, and display it on the UI 252. In some cases, the processors 254 may generate a healthy score as an evaluation of the user’s meal configuration, and/or recommendations on reducing/adding certain types of food.

In some embodiments, the dual-purpose computing device 250 may include one or more pre-trained machine learning models 256 for analyzing the sensor data collected by the precision weighing adapter 220 and the AI sensors 240.

In some embodiments, a weight pattern recognition machine learning (ML) model may be trained to receive sequences of weight readings from a plurality of load cell sensors 222 measuring weight changes occurred at the plurality of corners of a food container, embedding the sequences of weight readings into weight fluctuation patterns, identify (based on the weight fluctuation patterns) the compartment in the food container from which the user has taken food, and estimate the weight of the food taken by the user. The weight pattern recognition ML model may be trained with training data collected from experiments, simulations, and generated based on domain knowledge. Each piece of training data may include historical weight readings from load cell sensors, and labels indicating the actual compartment from which the food was taken and the weight of the food that was taken. In some embodiments, the weight pattern recognition ML model may be a neural network comprising an embedding layer for embedding the sequences of weight readings from the plurality of load cell sensors 222 into weight fluctuation patterns.

In some embodiments, a food recognition ML model may be trained for food identification based on the visual data collected by the AI cameras 244. The food recognition ML model may be implemented as a neural network comprising one or more feature extraction layers extracting features of the visual data (e.g., 3D images), and an output layer generating predictions of the food based on the extracted features. The visual data may include the images of the food stored in each compartment of a food container. The neural network may be trained based on labeled images of different types of food. In some embodiments, other types of sensors may be installed to help the food recognition ML model identify the food, such as olfactory receptors (also called smell receptors) and taste sensors.

In some embodiments, a user action recognition ML model may be trained for user action recognition based on the visual data collected by the AI cameras 244. The visual data may include 3D point cloud images of the users with depth information. In some cases, the visual data may focus on the hand motions or trajectories of the user. In some embodiments, the user action recognition ML model may include a channel-separated neural network, which captures spatial and spatiotemporal features in distinct layers. These features are separately captured by different convolution blocks but aggregated at each stage of convolution. By identifying the user actions, the IoT system may compensate or make corrections to the weight readings from the load cell sensors 222. For instance, when a user lifts a utensil from a compartment, the weight readings may detect the weight reduction and the above-described weight pattern recognition ML model may predict a certain amount of food has been taken from that compartment. However, the user action recognized by the user action recognition ML model, i.e., the user takes the utensil at this timestamp, may allow the computing device to adjust the output of the above-described weight pattern recognition ML model.

In some embodiments, the cloud server 260 may be implemented as the centralized brain that collects all the data from different foodservice providers and performs operational analytics, such as extracting trends in food consumption in certain areas, making predictions for future market needs, and proactively managing inventory replenishment, etc. The cloud server 260 may be configured in a multi-tenant mode to serve different foodservice providers at the same time. The cloud server 260 may also receive notices or error messages of malfunctioning sensors, and send out notices and instructions to local operators for repair.

FIG. 3 illustrates an exemplary portable IoT device 300 for IoT-based dietary tracking in accordance with some embodiments. The components and operations shown in FIG. 3 are for illustrative purposes only, and are not intended to limit the implementation of the portable IoT device 300. The portable IoT device 300 is designed to be used in venues like homes or offices, where installing the multi-sensor IoT system described in FIGS. 2A-2B is not practical. In some embodiments, the portable IoT device 300 may be designed as an electronic appliance like a coffee machine.

In some embodiments, the portable IoT device 300 may include a scale 310 coupled with one or more weight sensors, a first camera 320 facing the scale, a second camera 325 facing users, a built-in touch screen display 360 for I/O, and one or more processors 330 configured with a non-transient storage medium for storing and processing the data collected by the scale and cameras.

As shown in FIG. 3, the processors 330 may receive a first weight signal from the one or more weight sensors when a first user places food on the scale 310 at a first point in time at step 332. For instance, before a user starts to consume food, he/she may place the food on the scale 310. The food may be packed or unpacked. Then the processors 330 may receive a first image of the food placed by the first user on the scale from the first camera 320 facing the scale at step 334. If the food is still in a package, the first image may capture a barcode or a text on the package. Based on the barcode or text, the food may be identified with information including ingredients, allergen, nutrition, etc. If the food is unpacked, the first image may include a 2D or 3D image of the food. The image may be fed into a food image recognition machine learning model 350 for recognition at step 336.

The food image recognition machine learning model 350 may be trained by supervised learning algorithms based on a large number of training food images and proper labels. Based on the input image, the food image recognition machine learning model 350 may output one or more images of similar food for the user to confirm, select, and/or edit. For instance, if a user places a donut on the scale, the food image recognition machine learning model 350 may display several images of similar donuts on the built-in touch screen display 360 for the user to confirm. The user may select one of the displayed images that is the same as or closest to the actual donut. After the selection, the built-in touch screen display 360 may switch to another page to display detailed information of the donut on the selected image and provide the user options to edit the information.

After obtaining the food information of the food on the scale, the processors 330 may further determine the portion-based food information based on the first weight signal and the food information at step 338. For instance, the food information may include nutrition information (e.g., protein, calories, sugar, carbohydrate) per weight unit such as per gram or oz. The processors 330 may determine the portion-based food information as a product of the weight and the food information. The portion-based food information may accurately reflect the nutrition intake of the user based on the precise weight of the food consumed by the user.

In some embodiments, the second camera 325 may capture an image of the user at step 340. The image may be fed into a face recognition machine learning model 352 to identify the user at step 342. The face recognition machine learning model 352 may be trained by the user labeling previously captured or registered faces. For example, during the initial setup of the portable IoT device 300, multiple users may register their faces using the second camera 325. After identifying the user, the previously determined portion-based food information may be associated with the user (through the identification of the user) and displayed on the built-in touch screen display 360 at step 344.

In some embodiments, the identification of the user may be obtained through other means, such as using the user’s biometric features (e.g., fingerprint, voice). For instance, the built-in touch screen display 360 may include an in-screen fingerprint sensor for scanning a user’s fingerprint. The user’s fingerprint may be pre-registered with the portable IoT device 300 and associated with the user’s identification. As another example, since the IoT device 300 may only serve a limited number of users (family members in the same household), it may list the pre-registered user profiles on the built-in touch screen display 360 for the user to confirm his/her identification by selecting the corresponding profile.

In some embodiments, the determined portion-based food information and the corresponding user identification may be temporarily stored in the IoT device 300 before being sent to a cloud server 370 for dietary analysis and tracking for the user. This temporarily stored information may be subject to later adjustment for improving accuracy. In many scenarios, if the user does not finish the food, he/she may place the left-over on the scale 310 again to update the portion-based food information. The IoT device processors 330 may automatically detect that the same user does not finish the same food, and update the portion-based food information accordingly.

For instance, when a new user places food on the scale 310, the second camera 325 may capture an image of the new user. The image of the new user may be fed into the face recognition ML model 352 to obtain an identification of the new user. If the identification of the new user matches with the original user (i.e., it is the same user), the processors 330 may display on the display 360 a prompt for the user to confirm whether the food is new food or leftover. If the user confirms that the food is leftover, the portion-based dietary information may be updated based on a weight difference between the weight of the original food (e.g., measured before consumption) and the weight of the leftover (e.g., measured after consumption).

In particular, a second weight signal from the one or more weight sensors when the user places the left-over food on the scale 310 at a second point in time, and the first camera 320 may capture a second image of the food placed by the user on the scale from the first camera facing the scale. Based on the second image of the left-over food, the processors 330 may determine food information of the left-over food using the food image recognition ML model 350. If the processors 330 determine that the left-over food is similar to the original food (using the food image recognition ML model 350), the temporarily stored portion-based food information may be updated based on a difference between the first weight signal and the second weight signal. On the other hand, if the user confirms that the food is new through the prompt, the temporarily stored portion-based dietary information may be updated based on a sum of the first weight signal and the second weight signal. Furthermore, if the food image recognition ML model 350 determines that the new food is different from the original food, the processors 330 may skip the displaying of the prompt because the new food is unlikely to be leftover. In this case, the temporarily stored portion-based dietary information may be similarly updated based on a sum of the first weight signal and the second weight signal.

In some embodiments, the IoT device 300 may send the updated portion-based food information, the identification of the user, the first point in time, and the second point in time to the cloud server 370 for dietary tracking of the user’s food intake events. The time points may be used to determine the speed of food intake, which may be one of the factors defining a user’s dietary behavior. The updated portion-based information may be sent to the server after being stored locally for a preconfigured amount of time.

In some embodiments, the processors 330 may also be configured to: receive a second weight signal from the scale 310 when a second user places food on the scale at a second point in time; receive an image of the second user from the second camera 325; determine whether the second user matches the first user based on the image of the second user using the face recognition ML model 352. If the second user matches the first user, the processors 330 may receive a second image of the food placed by the second user on the scale 310 from the first camera 320 facing the scale; determine food information of the food placed by the second user based on the second image of the food using the food image recognition ML model 350; and determine whether a difference between the food information of the food placed by the first user and the food information of the food placed by the second user is within a preset threshold. In response to the difference between the food information of the food placed by the first user and the food information of the food placed by the second user being within the preset threshold, the processors 330 may display a notification message on the built-in touch screen display 360 querying if the food placed by the second user is leftover or new food. In response to the selection being leftover, the processors 330 may update the portion-based dietary information based on a difference between the first weight signal and the second weight signal. In response to the selection being new food, the processors 330 may update the portion-based dietary information based on a sum of the first weight signal and the second weight signal; and display the updated portion-based dietary information on the built-in touch screen display.

In some embodiments, in response to the difference between the food information of the food placed by the first user and the food information of the food placed by the second user being greater than the preset threshold (e.g., it is more likely that the food placed by the second user is not a left-over but a new food), the processors 330 may determine second portion-based dietary information of the food placed by the second user based on the food information of the food placed by the second user and the second weight signal; and display the second portion-based dietary information on the built-in touch screen display 360.

FIG. 4 illustrates an exemplary method of a mobile application 400 for IoT-based dietary tracking in accordance with some embodiments. The operations performed by the mobile application 400 in FIG. 4 are for illustrative purposes only, and may include more, fewer, or alternative operations depending on the implementation. The mobile application 400 may be used to collect user dietary activities when the user is in an environment without access to the multi-sensor IoT system described in FIGS. 2A-2B or the portable IoT device 300 described in FIG. 3.

In some embodiments, the mobile application 400 may determine a probability of a user conducting a food-intake activity based on spatial and temporal information, and send a notification message to the user’s mobile device for the user to respond. For instance, the mobile application 400 may receive a location signal and a time from the user’s mobile phone. The location signal may refer to GPS coordinates. The location signal and the time may be fed into a neural network to obtain a likelihood of a food intake event occurring at a location corresponding to the location signal and the time. The neural network may be trained based on a plurality of historical food intake events recorded by a plurality of users from the same region as the user, each food intake event comprising location information and time information. For instance, if a user walks through a park in the afternoon, the likelihood for him to have a food-intake event is low; if the user walks through a park at noon, and stays at one spot in the part for 10-15 minutes, the likelihood for him to having a food-intake event may increase. As another example, if a user approaches a restaurant during business hours, the likelihood for him to have a food-intake event may be high, regardless of what time it is. The neural network may be trained to learn the weights of different locations and time slots in determining the likelihood of a user having a food-intake event.

In some embodiments, in response to the notification message, the user may input information such as a scanned receipt, an image of food, a scanned QR code, or a payment made using the mobile device. For example, the image of the food that he/she is consuming may be fed into a food image recognition machine learning model for identification. The model may be trained with a large number of food images with proper labels. The label may include food name, nutrition information, price, etc. As another example, the scanned receipt or an image of the receipt may include the name of the food order by the user as well as the name of the foodservice provider. By using Optical Character Recognition (OCR), the text on the image may be extracted, which may be further processed using Natural Language Processing (NPL) to identify the name of the food and the service provider. Based on that information, the food information of the food on the receipt may be obtained from a backend database. The mobile application 400 may further allow the user to enter a portion size of the food that he/she consumed. The portion size and the food information may be used to determine the portion-based food information for the user. In some embodiments, the mobile application 400 may send such portion-based food information and a device identifier of the mobile device (e.g., or a user ID used to register the mobile application 400) to a cloud server for data aggregation and analysis, as well as dietary tracking for the user.

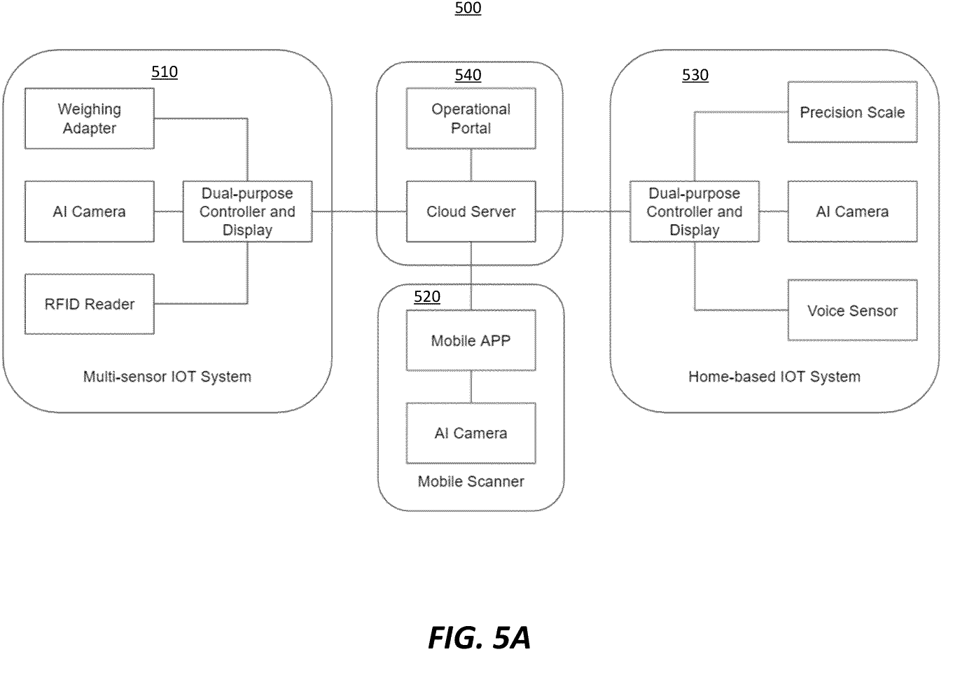

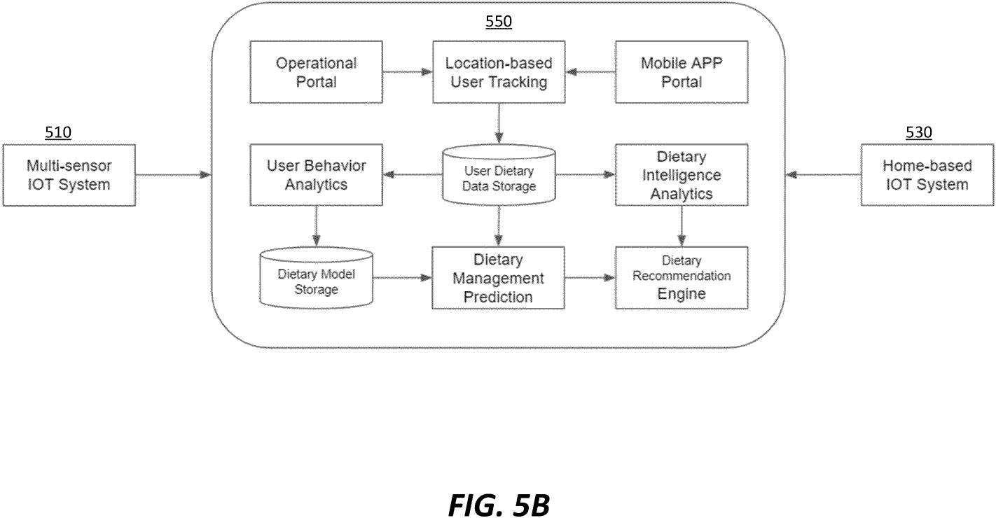

FIG. 5A illustrates an exemplary system diagram for IoT-based dietary tracking in accordance with some embodiments. As shown, the exemplary system may include multiple data-collecting devices that provide complete coverage of a user’s food-intake activities. These data collecting devices may include a multi-sensor IoT system 510, a home-based IoT system/device 530, and a mobile scanner 520 (e.g., a mobile phone with camera functionality). These data collecting devices may be installed or used in different settings.

For instance, the multi-sensor IoT system 510 has multiple hardware pieces to be installed and is thus more suitable for restaurants, hotels, cafeterias, or other foodservice establishments with relatively stationary configurations. The multi-sensor IoT system 510 may track customers’ food intake automatically without user involvement. In certain cases, the user may merely need to scan his ID card/RFID card so that the automatically collected dietary information can be linked to the user. In other cases, with facial recognition techniques, the user may be automatically identified and linked to his/her dietary information.

As another example, the home-based IoT system/device 530 may be designed as an appliance to be used at home or in the office. These settings may have limited spaces and will unlike serve a large number of users, therefore they may not be suitable for installing the more complete and powerful but relatively complicated multi-sensor IoT system 510. An exemplary architecture of the home-based IoT system/device 530 may be found in FIG. 3. In some embodiments, the home-based IoT system/device 530 may further include a voice sensor as both an input and output portal. For example, the user may issue voice commands to the home-based IoT system/device 530 through the voice sensor or receive notifications or feedback from the home-based IoT system/device 530 through the voice sensor.

As yet another example, the mobile scanner 520 may refer to the mobile device installed with the mobile application described in FIG. 4. The mobile scanner 520 may be designed to collect the food-intake activities that cannot be tracked by the multi-sensor IoT system 510 or the home-based IoT system/device 530. For example, when a user eats in the park, near a food truck, or in a restaurant without the multi-sensor IoT system 510, he/she may use the mobile scanner to automatically identify and upload the food information.

The data collected by these data-collecting devices 510, 520, and 530 may be aggregated at a cloud server 540 for further process. The cloud server 540 may collect dietary events of a large number of users for a period of time, learn the trends and patterns underlying the data, and make dietary recommendations or reports to the users. In some embodiments, the cloud server 540 may include an operational portal for operators to configure the data collection devices, define the data analysis methods, feed training data for the cloud server 540 to train various machine learning models, etc.

In some embodiments, the cloud server 540 may first cluster the dietary records of each user, and extract a plurality of dietary features of the user based on the records. The plurality of dietary features may have redundancy and not be equally informative for differentiating various dietary behaviors. To identify the smallest feature subset that best uncovers the latent correlations and distinctions among the dietary behavioral groups, feature selection techniques such as coefficient correlation may be used. Based on the feature subset, the users may be clustered into dietary behavioral groups. Each group includes the users having the same or similar dietary behavior.

In some embodiments, the dietary behavioral groups may be labeled in different ways. For instance, one or more users may provide their profile information during registration. The profile information may be used to label the entire group to which the one or more users belong. After labeling, the cloud server 540 may train a classification model using supervised learning based on the labeled dietary behavioral groups. The trained classification model may receive a plurality of dietary records of a new user and predict a dietary behavioral group to which the new user belongs. Based on this knowledge, the cloud server 540 may evaluate the new user’s dietary behavior against the population average of other users in the same dietary behavioral group. Based on the evaluation, the cloud server 540 may make recommendations to the new user.

In other embodiments, the cloud server 540 may also receive the dietary goals of the plurality of users, and cluster the users into a plurality of dietary goal groups (e.g., weight-loss group, body-building group, cholesterol level management group). When the dietary records and a dietary goal of a new user are uploaded, the cloud server 540 may evaluate the new user’s dietary behavior against the population average of other users in the same dietary goal group. The evaluation may reflect whether the new user is behind in certain aspects compared to the average values (e.g., the new users may intake too much carbohydrate than an average user in the same dietary goal group).

FIG. 5B illustrates another exemplary system diagram of a cloud server 550 for IoT-based dietary tracking in accordance with some embodiments. The cloud server 550 may communicate with other data collecting devices such as multi-sensor IoT system 510 and home-based IoT system 530 to aggregate user dietary records. In some embodiments, the cloud server 550 may also receive dietary records from mobile applications through a mobile application portal. The mobile application may perform location-based user tracking to remind the user to track his/her dietary activities. With these different types of data sources, the cloud server 550 may achieve complete coverage of users’ dietary data tracking.

As shown in FIG. 550, the cloud server 550 may include a user dietary data storage for storing the received dietary records of a plurality of users for further analysis, including user dietary behavior analysis, dietary intelligence analysis, dietary goal management, and dietary recommendation analysis, etc. The cloud server 550 may also include a dietary machine learning model storage for storing trained machine learning models. As described in FIG. 5A, the trained user classification model may be stored in the dietary model storage. In some embodiments, after classifying a user into a user dietary behavioral group, the classification result may be displayed to the user. The user may be offered an option to correct the classification. If the user corrects the classification, the user classification model may automatically record the user’s dietary behavior features and the correct classification (label) as new training data. Once the number of new training data reaches a threshold, the user classification model may retrain itself to improve accuracy for future users.

One of the challenges in consumers’ end-to-end or complete coverage of dietary behavior is how to correlate the plurality of food-intake events collected from different sources. For instance, a user may grab breakfast at a coffee shop, have lunch at a workplace cafeteria installed with the above-described IoT system (in FIGS. 2A and 2B), and eat dinner at home equipped with the above-described portable IoT device (in FIG. 3). The IoT system in the cafeteria, the IoT device at home, and the mobile application (e.g., used to track the breakfast in the coffee shop) may not be able to correlate the respectively collected food intake events to the same user.

In some embodiments, a correlation process using geometrically distributed IoT sensors may be executed to correlate the food intake events collected from the different devices/applications at different venues.

For instance, when a user approaches a food service where a multi-sensor IoT system 510 is deployed, one or more sensors of the IoT system 510 may detect the presence of the user’s mobile device, which may trigger a notification to be sent to the mobile application installed on the user’s mobile device. The user may process the notification by scanning a QR code generated by the IoT system 510 and presented on the IoT sensor display. Wherein the QR code may include a unique identifier, and the scanning of the QR code may associate the identification of the user with the unique identifier. When the user takes food from the food service, the IoT system 510 may have geometrically distributed sensors to track the user activity (e.g., using geometrically distributed sensors coupled to a scale), identify the food taken by the user (e.g., using geometrically distributed cameras to rebuild the 3D image of the food and identifying the food based on the 3D image), determine the portion sizes of the food, and calculate the portion-based dietary information. The portion-based dietary information may be associated with the identification of the user through the unique identifier included in the QR code. The combination of the portion-based dietary information and the user identification may be sent to the cloud server 550 for user dietary records correlation. In some embodiments, the notification may prompt the user to scan a receipt, configure his worker identification with the IoT system 510, or use the mobile application to make a payment. These actions will register the user’s identification with the IoT system 510, which may be used to associate with the automatically detected food intake event of the user. In other group-meal settings where one person is paying for a group of people, each individual user may receive the above-described notification on his/her mobile device to confirm identification.

As another example, the user may pre-register his/her identification with the IoT device at home or office. While the IoT device detects portion-based dietary information, it may obtain the user’s identification based on the user’s biometric features (e.g., through facial recognition, fingerprint scan, voice recognition) or user’s selection of a list of user profiles. When uploading to the cloud server 550, the user’s identification may be associated with the detected portion-based dietary information. As yet another example, the user’s identification may also be pre-registered with the mobile application installed on the user’s mobile device, so that the user’s identification is attached to the dietary event uploaded by the user using his/her mobile file,

In other embodiments, the users may voluntarily agree to have their identification information stored by the IoT system (e.g., worker/employee ID, payment ID, biometric features), so that future food intake events may be automatically associated with the user without sending/processing the above-described notifications.

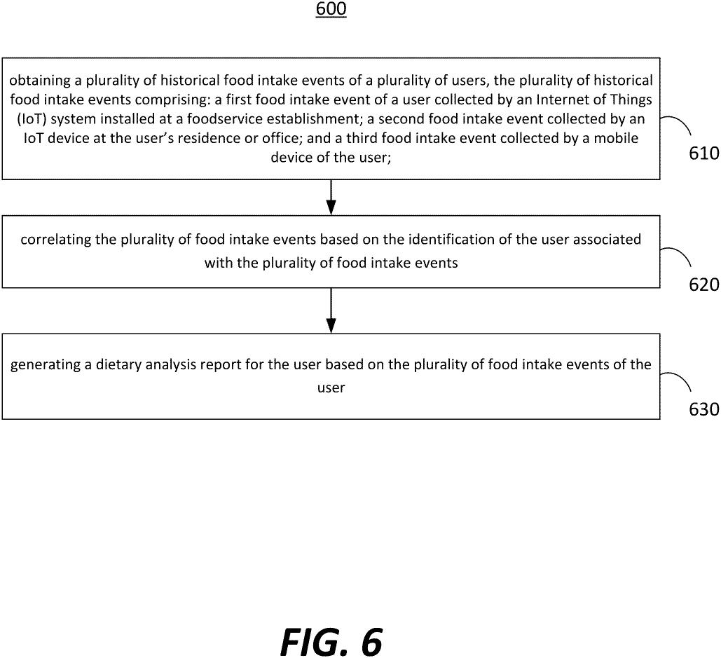

FIG. 6 illustrates an exemplary method 600 for IoT-based dietary tracking in accordance with some embodiments. Method 600 may be performed by a computer device, apparatus, or system. The method 600 may be performed by one or more modules/components of the environment or system illustrated by FIGS. 1-5B. The operations of the method 600 presented below are intended to be illustrative. Depending on the implementation, the method 600 may include additional, fewer, or alternative steps performed in various orders or parallel.

Block 610 includes collecting a plurality of historical food intake events of a plurality of users, the plurality of historical food intake events comprising: a first food intake event of a user collected by an Internet of Things (IoT) system installed at a foodservice establishment; a second food intake event collected by an IoT device at the user’s residence or office; and a third food intake event collected by a mobile device of the user.

In some embodiments, the collecting of the first food intake event may include determining portion-based dietary information by monitoring the user’s food taking actions using geometrically distributed sensors, wherein the geometrically distributed sensors comprise distributed weight sensors attached to a scale and one or more cameras; generating a notification on a mobile device of the user for confirming an identification of the user; and associating the portion-based dietary information of the first food intake event with the user’s identity to form a first food intake event.

In some embodiments, the electronic appliance comprises: a scale coupled with one or more weight sensors and a first camera facing the scale. The collecting of the second food intake event may include receiving a first weight signal from the one or more weight sensors when the user places first food on the scale; receiving an image of the first food from the first camera facing the scale; determining food information of the first food based on the image of the first food using a first machine learning model for food image recognition; determining portion-based dietary information of the first food based on the food information of the first food and the first weight signal; determining the identification of the user based on the user’s biometric features or the user’s selection from a list of user profiles; and associating the identification of the user with the portion-based dietary information of the first food to form a second food intake event.

Block 620 includes correlating the plurality of food intake events based on the identification of the user associated with the plurality of food intake events.

Block 630 includes generating a dietary analysis report for the user based on the plurality of food intake events of the user.

In some embodiments, the collecting the second food intake events of the user using the electronic appliance further comprises: when another user places second food on the scale, receiving a second weight signal from the one or more weight sensors; receiving an identification of the another user; in response to the identification of the another user being the same as the identification of the user, displaying, on a display of the electronic appliance, a prompt for the user to confirm whether the second food is new food or leftover; in response to the second food being leftover, updating the portion-based dietary information based on a difference between the first weight signal and the second weight signal; and in response to the second food being new food, updating the portion-based dietary information based on a sum of the first weight signal and the second weight signal.

In some embodiments, the collecting the second food intake events of the user using the electronic appliance further comprises: when the user places second food on the scale, receiving an image of the second food from the first camera and a second weight signal from the one or more weight sensors; determining whether the second food is same as the first food using the first machine learning model based on the image of the second food; if the second food is the same as the first food, displaying, on a display of the electronic appliance, a prompt for the user to confirm whether the second food is new food or leftover; in response to the second food being leftover, updating the portion-based dietary information based on a difference between the first weight signal and the second weight signal; and in response to the second food being new food, updating the portion-based dietary information based on a sum of the first weight signal and the second weight signal.

In some embodiments, the collecting the second food intake events of the user using the electronic appliance further comprises: in response to determining that the second food is different from the first food, updating the portion-based dietary information based on the sum of the first weight signal and the second weight signal.

In some embodiments, the collecting the second food intake events of the user using the electronic appliance further comprises: associating the identification of the user, the portion-based dietary information of the first food, and a current timestamp to form the second food intake event.

In some embodiments, the generating of the dietary analysis report for the user comprises: receiving a request comprising a time window; and generating a list of food intake events of the user between the time window, wherein each food intake event comprises a time, a location, and portion-based food information of food taken by the user.

In some embodiments, the determining the set of dietary features of each of the plurality of users based on the plurality of historical food intake events comprises: determining a plurality of features based on the plurality of historical food intake events of the plurality of users; determining a correlation coefficient between each pair of the plurality of features; grouping the plurality of features based on the correlation coefficients into one or more groups; and selecting one feature from each of the one or more groups to form the set of dietary features.

In some embodiments, the generating of the dietary analysis report for the user comprises: receiving a plurality of historical dietary goals from a plurality of users; clustering the plurality of users based on the plurality of historical dietary goals into a plurality of dietary goal groups; and for each of the plurality of dietary goal groups, determining representative dietary features values for the dietary goal group based on a set of dietary features of the users in the dietary goal group.

In some embodiments, the generating of the dietary analysis report for the user comprises: obtaining a plurality of historical food intake events of a plurality of users; determining, using feature selection techniques in machine learning, a set of dietary features of each of the plurality of users based on the plurality of historical food intake events; clustering, using unsupervised learning, the plurality of users into a plurality of dietary behavioral groups based on the set of dietary features of each of the plurality of users; obtaining a plurality of group labels for the plurality of dietary behavioral groups; training, using supervised training, a classification model based on the plurality of group labels and the set of dietary features; classifying, using the classification model, the user into one of the plurality of dietary behavioral groups based on the set of dietary features of the user extracted from the plurality of food intake events of the user; and generating the dietary analysis report for the user based on the set of dietary features of the user and other users in the classified dietary behavioral group.

In some embodiments, the collecting the first food intake event of the user using the IoT system installed at the foodservice establishment comprises: receiving the first food intake event of the user from the IoT system, wherein the first food intake event comprises a time, a location, portion-based food information of food taken by the user at the foodservice establishment, and an identification of the user.

In some embodiments, the collecting the third food intake events comprises: installing an application on the mobile device of the user, wherein the application comprises a trained machine learning model that is trained to receive a food image and output one or more predicted food images that are similar to the food image; and receiving, from the application, a third food intake event comprising a time, a user selection of the one or more predicted food images generated by the trained machine learning model, and an identification of the user.

In some embodiments, the method 600 may further include detecting dietary behavioral change by comparing the plurality of food intake events of the user against a plurality of historical food intake events of the user; determining, based on the dietary behavioral change, one or more probabilities that the user is moving from one dietary behavioral group to one or more other dietary behavioral groups; and generating a prediction report for the user based on a highest probability from the one or more probabilities.

In some embodiments, the electronic appliance further comprises a second camera facing users, and the determining of the identification of the user comprises: receiving an image of the user from the second camera; obtaining an identification of the user based on the image of the user using a second machine learning model for face recognition.

In some embodiments, the determining of the identification of the user comprises: displaying a prompt comprising a list of user profiles that have registered with the electronic appliance; and receiving a selection from the list of user profiles as the identification of the user.

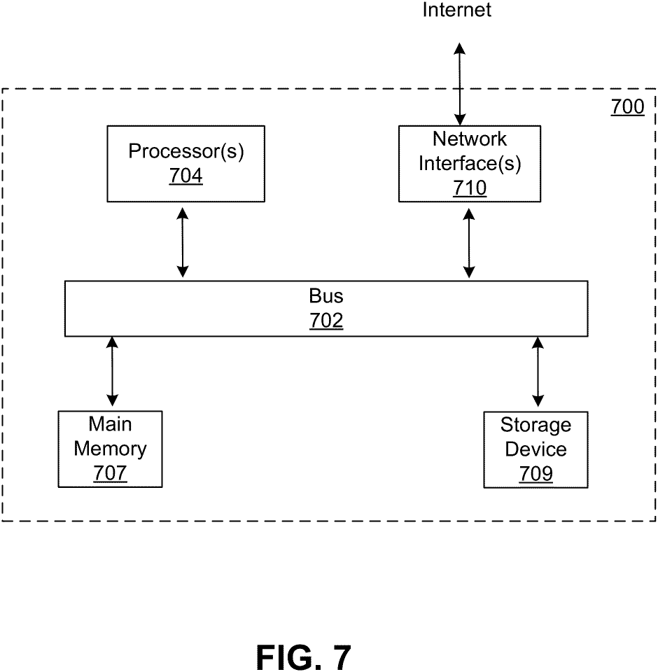

FIG. 7 illustrates an example computing device in which any of the embodiments described herein may be implemented. The computing device may be used to implement one or more components of the systems and the methods shown in FIGS. 1A-6 The computing device 700 may comprise a bus 702 or other communication mechanism for communicating information and one or more hardware processors 704 coupled with bus 702 for processing information. Hardware processor(s) 704 may be, for example, one or more general purpose microprocessors.

The computing device 700 may also include a main memory 707, such as a random-access memory (RAM), cache and/or other dynamic storage devices, coupled to bus 702 for storing information and instructions to be executed by processor(s) 704. Main memory 707 also may be used for storing temporary variables or other intermediate information during execution of instructions to be executed by processor(s) 704. Such instructions, when stored in storage media accessible to processor(s) 704, may render computing device 700 into a special-purpose machine that is customized to perform the operations specified in the instructions. Main memory 707 may include non-volatile media and/or volatile media. Non-volatile media may include, for example, optical or magnetic disks. Volatile media may include dynamic memory. Common forms of media may include, for example, a floppy disk, a flexible disk, hard disk, solid state drive, magnetic tape, or any other magnetic data storage medium, a CD-ROM, any other optical data storage medium, any physical medium with patterns of holes, a RAM, a DRAM, a PROM, and EPROM, a FLASH-EPROM, NVRAM, any other memory chip or cartridge, or networked versions of the same.

The computing device 700 may implement the techniques described herein using customized hard-wired logic, one or more ASICs or FPGAs, firmware and/or program logic which in combination with the computing device may cause or program computing device 700 to be a special-purpose machine. According to one embodiment, the techniques herein are performed by computing device 700 in response to processor(s) 704 executing one or more sequences of one or more instructions contained in main memory 707. Such instructions may be read into main memory 707 from another storage medium, such as storage device 709. Execution of the sequences of instructions contained in main memory 707 may cause processor(s) 704 to perform the process steps described herein. For example, the processes/methods disclosed herein may be implemented by computer program instructions stored in main memory 707. When these instructions are executed by processor(s) 704, they may perform the steps as shown in corresponding figures and described above. In alternative embodiments, hard-wired circuitry may be used in place of or in combination with software instructions.

The computing device 700 also includes a communication interface 710 coupled to bus 702. Communication interface 710 may provide a two-way data communication coupling to one or more network links that are connected to one or more networks. As another example, communication interface 710 may be a local area network (LAN) card to provide a data communication connection to a compatible LAN (or WAN component to communicate with a WAN). Wireless links may also be implemented.

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

Each process, method, and algorithm described in the preceding sections may be embodied in, and fully or partially automated by, code modules executed by one or more computer systems or computer processors comprising computer hardware. The processes and algorithms may be implemented partially or wholly in application-specific circuitry.

When the functions disclosed herein are implemented in the form of software functional units and sold or used as independent products, they can be stored in a processor-executable non-volatile computer-readable storage medium. Particular technical solutions disclosed herein (in whole or in part) or aspects that contribute to current technologies may be embodied in the form of a software product. The software product may be stored in a storage medium, comprising a number of instructions to cause a computing device (which may be a personal computer, a server, a network device, and the like) to execute all or some steps of the methods of the embodiments of the present application. The storage medium may comprise a flash drive, a portable hard drive, ROM, RAM, a magnetic disk, an optical disc, another medium operable to store program code, or any combination thereof.

Particular embodiments further provide a system comprising a processor and a non-transitory computer-readable storage medium storing instructions executable by the processor to cause the system to perform operations corresponding to steps in any method of the embodiments disclosed above. Particular embodiments further provide a non-transitory computer-readable storage medium configured with instructions executable by one or more processors to cause the one or more processors to perform operations corresponding to steps in any method of the embodiments disclosed above.

Embodiments disclosed herein may be implemented through a cloud platform, a server or a server group (hereinafter collectively the “service system”) that interacts with a client. The client may be a terminal device, or a client registered by a user at a platform, wherein the terminal device may be a mobile terminal, a personal computer (PC), and any device that may be installed with a platform application program.

The various features and processes described above may be used independently of one another or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The exemplary systems and components described herein may be configured differently than described. For example, elements may be added to, removed from, or rearranged compared to the disclosed example embodiments.

The various operations of exemplary methods described herein may be performed, at least partially, by an algorithm. The algorithm may be comprised in program codes or instructions stored in a memory (e.g., a non-transitory computer-readable storage medium described above). Such algorithm may comprise a machine learning algorithm. In some embodiments, a machine learning algorithm may not explicitly program computers to perform a function but can learn from training data to make a prediction model that performs the function.

The various operations of exemplary methods described herein may be performed, at least partially, by one or more processors that are temporarily configured (e.g., by software) or permanently configured to perform the relevant operations. Whether temporarily or permanently configured, such processors may constitute processor-implemented engines that operate to perform one or more operations or functions described herein.

Similarly, the methods described herein may be at least partially processor-implemented, with a particular processor or processors being an example of hardware. For example, at least some of the operations of a method may be performed by one or more processors or processor-implemented engines. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

As used herein, “or” is inclusive and not exclusive, unless expressly indicated otherwise or indicated otherwise by context. Therefore, herein, “A, B, or C” means “A, B, A and B, A and C, B and C, or A, B, and C,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context. Moreover, plural instances may be provided for resources, operations, or structures described herein as a single instance. Additionally, boundaries between various resources, operations, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

The term “include” or “comprise” is used to indicate the existence of the subsequently declared features, but it does not exclude the addition of other features. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

What is claimed is:

1. A computer-implemented method, comprising:
obtaining a plurality of food intake events of a user occurred at a plurality of venues, wherein the obtaining comprises:
collecting first food intake events of the user using an Internet of Things (IoT) system installed as a foodservice establishment, wherein the collecting comprises:
determining portion-based dietary information by monitoring the user’s food taking actions using geometrically distributed sensors, wherein the geometrically distributed sensors comprise weight sensors and cameras;
generating a notification on a mobile device of the user for confirming an identification of the user; and
associating the portion-based dietary information of the first food intake event with the user’s identity to form a first food intake event;
collecting second food intake events of the user using an electronic appliance placed at the user’s residence or office, wherein the electronic appliance comprises: a scale coupled with one or more weight sensors and a first camera facing the scale, and wherein the collecting comprises:
receiving a first weight signal from the one or more weight sensors when the user places first food on the scale;
receiving an image of the first food from the first camera facing the scale;
determining food information of the first food based on the image of the first food using a first machine learning model for food image recognition;
determining portion-based dietary information of the first food based on the food information of the first food and the first weight signal;
determining the identification of the user based on the user’s biometric features or the user’s selection from a list of user profiles; and
associating the identification of the user with the portion-based dietary information of the first food to form a second food intake event;
collecting third food intake events of the user using a mobile application installed on the mobile device of the user;
correlating the plurality of food intake events based on the identification of the user associated with the plurality of food intake events; and
generating a dietary analysis report for the user based on the plurality of food intake events of the user.

2. The method of claim 1, wherein the collecting the second food intake events of the user using the electronic appliance further comprises:
when another user places second food on the scale, receiving a second weight signal from the one or more weight sensors;
receiving an identification of the another user;
in response to the identification of the another user being the same as the identification of the user, displaying, on a display of the electronic appliance, a prompt for the user to confirm whether the second food is new food or leftover;
in response to the second food being leftover, updating the portion-based dietary information based on a difference between the first weight signal and the second weight signal; and
in response to the second food being new food, updating the portion-based dietary information based on a sum of the first weight signal and the second weight signal.

3. The method of claim 1, wherein the collecting the second food intake events of the user using the electronic appliance further comprises:
when the user places second food on the scale, receiving an image of the second food from the first camera and a second weight signal from the one or more weight sensors;
determining whether the second food is same as the first food using the first machine learning model based on the image of the second food;
if the second food is the same as the first food, displaying, on a display of the electronic appliance, a prompt for the user to confirm whether the second food is new food or leftover;
in response to the second food being leftover, updating the portion-based dietary information based on a difference between the first weight signal and the second weight signal; and
in response to the second food being new food, updating the portion-based dietary information based on a sum of the first weight signal and the second weight signal.

4. The method of claim 3, wherein the collecting the second food intake events of the user using the electronic appliance further comprises:
in response to determining that the second food is different from the first food, updating the portion-based dietary information based on the sum of the first weight signal and the second weight signal.

5. The method of claim 1, wherein the collecting the second food intake events of the user using the electronic appliance further comprises:
associating the identification of the user, the portion-based dietary information of the first food, and a current timestamp to form the second food intake event.

6. The method of claim 1, wherein the generating of the dietary analysis report for the user comprises:
receiving a request comprising a time window; and
generating a list of food intake events of the user between the time window, wherein each food intake event comprises a time, a location, and portion-based food information of food taken by the user.

7. The method of claim 1, wherein the generating of the dietary analysis report for the user comprises:
obtaining a plurality of historical food intake events of a plurality of users;
determining, using feature selection techniques in machine learning, a set of dietary features of each of the plurality of users based on the plurality of historical food intake events;
clustering, using unsupervised learning, the plurality of users into a plurality of dietary behavioral groups based on the set of dietary features of each of the plurality of users;
obtaining a plurality of group labels for the plurality of dietary behavioral groups;
training, using supervised training, a classification model based on the plurality of group labels and the set of dietary features;
classifying, using the classification model, the user into one of the plurality of dietary behavioral groups based on the set of dietary features of the user extracted from the plurality of food intake events of the user; and
generating the dietary analysis report for the user based on the set of dietary features of the user and other users in the classified dietary behavioral group.

8. The method of claim 1, wherein the generating of the dietary analysis report for the user comprises:
receiving a plurality of historical dietary goals from a plurality of users;
clustering the plurality of users based on the plurality of historical dietary goals into a plurality of dietary goal groups; and
for each of the plurality of dietary goal groups, determining representative dietary features values for the dietary goal group based on a set of dietary features of the users in the dietary goal group.

9. The method of claim 8, wherein the generating of the dietary analysis report for the user comprises:
receiving a dietary goal from the user;
identifying one of the plurality of dietary goal groups to which the dietary goal of the user belongs;
determining a distance between the set of dietary features of the user and the representative feature values of the identified dietary goal group; and
generating the dietary analysis report for the new user based on the distance.

10. The method of claim 7, wherein the determining the set of dietary features of each of the plurality of users based on the plurality of historical food intake events comprises:
determining a plurality of features based on the plurality of historical food intake events of the plurality of users;
determining a correlation coefficient between each pair of the plurality of features;
grouping the plurality of features based on the correlation coefficients into one or more groups; and
selecting one feature from each of the one or more groups to form the set of dietary features.

11. The method of claim 1, wherein the collecting the first food intake event of the user using the IoT system installed at the foodservice establishment comprises:
receiving the first food intake event of the user from the IoT system, wherein the first food intake event comprises a time, a location, portion-based food information of food taken by the user at the foodservice establishment, and an identification of the user.

12. The method of claim 1, wherein the collecting the third food intake events comprises:
installing an application on the mobile device of the user, wherein the application comprises a trained machine learning model that is trained to receive a food image and output one or more predicted food images that are similar to the food image; and
receiving, from the application, a third food intake event comprising a time, a user selection of the one or more predicted food images generated by the trained machine learning model, and an identification of the user.

13. The method of claim 1, further comprising:
detecting dietary behavioral change by comparing the plurality of food intake events of the user against a plurality of historical food intake events of the user;
determining, based on the dietary behavioral change, one or more probabilities that the user is moving from one dietary behavioral group to one or more other dietary behavioral groups; and
generating a prediction report for the user based on a highest probability from the one or more probabilities.

14. The method of claim 1, wherein the electronic appliance further comprises a second camera facing users, and
the determining of the identification of the user comprises:
receiving an image of the user from the second camera;
obtaining an identification of the user based on the image of the user using a second machine learning model for face recognition.

15. The method of claim 1, wherein the determining of the identification of the user comprises:
displaying a prompt comprising a list of user profiles that have registered with the electronic appliance; and
receiving a selection from the list of user profiles as the identification of the user.

16. A system comprising one or more processors and one or more non-transitory computer-readable memories coupled to the one or more processors, the one or more non-transitory computer-readable memories storing instructions that, when executed by the one or more processors, cause the system to perform operations comprising:
collecting first food intake events of the user using an Internet of Things (IoT) system installed as a foodservice establishment, wherein the collecting comprises:
determining portion-based dietary information by monitoring the user’s food taking actions using geometrically distributed sensors, wherein the geometrically distributed sensors comprise weight sensors and cameras;
generating a notification on a mobile device of the user for confirming an identification of the user; and
associating the portion-based dietary information of the first food intake event with the identification of the user to form a first food intake event;
collecting second food intake events of the user using an electronic appliance placed at the user’s residence or office, wherein the electronic appliance comprises: a scale coupled with one or more weight sensors and a first camera facing the scale, and wherein the collecting comprises:
receiving a first weight signal from the one or more weight sensors when the user places first food on the scale;
receiving an image of the first food from the first camera facing the scale;
determining food information of the first food based on the image of the first food using a first machine learning model for food image recognition;
determining portion-based dietary information of the first food based on the food information of the first food and the first weight signal;
determining the identification of the user based on the user’s biometric features or the user’s selection from a list of user profiles; and
associating the identification of the user with the portion-based dietary information of the first food to form a second food intake event;
collecting third food intake events of the user using a mobile application installed on the mobile device of the user;
correlating the plurality of food intake events based on the identification of the user associated with the plurality of food intake events; and
generating a dietary analysis report for the user based on the plurality of food intake events of the user.

17. The system of claim 16, wherein the collecting the second food intake events of the user using the electronic appliance further comprises:
when another user places second food on the scale, receiving a second weight signal from the one or more weight sensors;
determining an identification of the another user;
in response to the identification of the another user being the same as the identification of the user, displaying, on a display of the electronic appliance, a prompt for the user to confirm whether the second food is new food or leftover;
in response to the second food being leftover, updating the portion-based dietary information based on a difference between the first weight signal and the second weight signal; and
in response to the second food being new food, updating the portion-based dietary information based on a sum of the first weight signal and the second weight signal.

18. The system of claim 16, wherein the collecting the second food intake events of the user using the electronic appliance further comprises:
when the user places second food on the scale, receiving an image of the second food from the first camera and a second weight signal from the one or more weight sensors;
determining whether the second food is same as the first food using the first machine learning model based on the image of the second food;
if the second food is the same as the first food, displaying, on a display of the electronic appliance, a prompt for the user to confirm whether the second food is new food or leftover;
in response to the second food being leftover, updating the portion-based dietary information based on a difference between the first weight signal and the second weight signal; and
in response to the second food being new food, updating the portion-based dietary information based on a sum of the first weight signal and the second weight signal.

19. The system of claim 18, wherein the obtaining the second food intake events of the user using the electronic appliance further comprises:
in response to determining that the second food is different from the first food, updating the portion-based dietary information based on the sum of the first weight signal and the second weight signal.

20. A non-transitory computer-readable storage medium, configured with instructions executable by one or more processors to cause the one or more processors to perform operations comprising:
collecting first food intake events of the user using an Internet of Things (IoT) system installed as a foodservice establishment, wherein the collecting comprises:
determining portion-based dietary information by monitoring the user’s food taking actions using geometrically distributed sensors, wherein the geometrically distributed sensors comprise weight sensors and cameras;
generating a notification on a mobile device of the user for confirming an identification of the user; and
associating the portion-based dietary information of the first food intake event with the identification of the user to form a first food intake event;
collecting second food intake events of the user using an electronic appliance placed at the user’s residence or office, wherein the electronic appliance comprises: a scale coupled with one or more weight sensors and a first camera facing the scale, and wherein the collecting comprises:
receiving a first weight signal from the one or more weight sensors when the user places first food on the scale;
receiving an image of the first food from the first camera facing the scale;
determining food information of the first food based on the image of the first food using a first machine learning model for food image recognition;
determining portion-based dietary information of the first food based on the food information of the first food and the first weight signal;
determining the identification of the user based on the user’s biometric features or the user’s selection from a list of user profiles; and
associating the identification of the user with the portion-based dietary information of the first food to form a second food intake event;
collecting third food intake events of the user using a mobile application installed on the mobile device of the user;
correlating the plurality of food intake events based on the identification of the user associated with the plurality of food intake events; and
generating a dietary analysis report for the user based on the plurality of food intake events of the user.
